(19) United States
(12) Patent Application Publication
(10) Pub. No.: US 2021/0244848 A1
(43) Pub. Date: Aug. 12, 2021

(54) SOLID SUSPENSION

(71) Applicant: THE UNIVERSITY OF SHEFFIELD, Sheffield (GB)

(72) Inventors: Caroline Joanne Harrison, Sheffield (GB); Paul Vincent Hatton, Sheffield (GB); Rebecca Louise Hutchinson, Sheffield (GB); Cheryl Ann Miller, Sheffield (GB)

(73) Assignee: THE UNIVERSITY OF SHEFFIELD, Sheffield (GB)

(21) Appl. No.: 17/271,107

(22) PCT Filed: Aug. 27, 2019
(86) PCT No.: PCT/GB2019/052385

§ 371 Date: Feb. 24, 2021

(30) Foreign Application Priority Data
Aug. 28, 2018 (GB) 1813928.7

(51) Int. Cl. A61L 27/04; A61L 27/12; A61L 27/58; A61L 27/10

(52) U.S. Cl. CPC A61L 27/047; A61L 27/12; A61L 27/58; A61L 27/10; A61L 2430/12; A61L 2400/06; A61L 2430/02; A61L 2430/38; A61L 2400/12

(57) ABSTRACT

A solid suspension for use in bone regeneration and/or the repair of bone defects, comprising a source of at least one group II metal cation and a source of zinc cations, wherein the source of zinc cations comprises zinc oxide and wherein where there is only one group II metal cation this is strontium. A bone graft comprising a solid suspension, a method of preparation of a solid suspension and a use of a solid suspension in bone regeneration and/or in the repair of bone defects.

The invention relates to a solid suspension for use in bone regeneration and/or the repair of bone defects, in particular to a solid suspension comprising a source of at least one group II metal cation and a source of zinc cations. The invention further relates to a bone graft, and to a method of preparing both the suspension and the bone graft.

The promotion of bone regeneration (osteogenesis) remains a significant challenge in orthopaedic and dental surgery. The relatively recent commercial manufacture of solid suspensions of hydroxyapatite has provided surgeons with an injectable biomaterial that promotes bone tissue regeneration. WO 98/18719 describes a hydroxyapatite paste with excellent homogeneity, and U.S. Pat. No. 7,387,785 describes nano-sized crystalline hydroxyapatite synthesis. A solid solution for use in bone tissue regeneration is described in WO 2017/137005, which discloses a bone regeneration solid solution with an optimised rate of calcium release. The solid solution is intended for use in the treatment of osteoporosis and comprises a combination of cations, including calcium, magnesium, strontium, barium and/or zinc.

It is desirable for these materials to have antimicrobial properties, ensuring that the solid suspension assists healing by preventing post-surgical infection. Silver-doped hydroxyapatite pastes have been found to offer one solution to this problem (Wilcock C J, Stafford G P, Miller C A, Ryabenkova Y, Fatima M, Gentile P, Mobus G & Hatton P V (2017) Preparation and Antibacterial Properties of Silver-Doped Nanoscale Hydroxyapatite Pastes for Bone Repair and Augmentation. Journal of Biomedical Nanotechnology, 13(9), 1168-1176).

However, it would be desirable to offer alternative materials, which can repair bone defects, ideally which have bio-regenerative and antimicrobial properties. The invention is intended to overcome or ameliorate at least some aspects of this problem.

Accordingly, in a first aspect of the invention there is provided a solid suspension for use in bone regeneration and/or the repair of bone defects, the suspension comprising a source of at least one group II metal cation and zinc cations, wherein the zinc cations are provided as zinc oxide. When there is only one group II metal cation this is generally strontium, although magnesium and calcium may also be the “one” group II metal cation. The solid suspension (“paste”) described herein has been found to have excellent antimicrobial properties. Without being bound by theory, this is believed to be as a result of the presence of both zinc and group II metal cations. It will often be the case that the group II metal cations are selected from calcium, strontium, magnesium and combinations thereof. Very often the group II metal cations will include calcium, strontium or a mixture of these. The presence of strontium has been found to be particularly beneficial in enhancing the antimicrobial properties of the suspension.

As used herein, the term bone regeneration is intended to refer to the growth of new bone at sites where the bone is weak, either through damage or insufficient growth. Bone regeneration may be required where, for instance, it is necessary to restore proper function of the bone, such as in the treatment of osteoporosis and osteoarthritis. The repair of bone defects, as referred to herein, is distinct from bone regeneration in that it refers to the repair of damaged or weakened bone sites through their prosthetic restoration, wherein the solid suspension of the invention is used to reconstruct the bone for instance, by filling holes or providing a physical barrier layer. Bone regeneration and the repair of bone defects will often occur together when the solid suspension of the invention is applied.

As used herein the term “solid suspension” is intended to mean a homogenous mixture or suspension of a solid in a liquid, such as a sol or paste. Whether the solid suspension is a sol or a paste is dependent upon the relative amount of liquid and solid in the suspension. Solid suspensions are distinct from glasses in that they contain a liquid phase, whereas glasses are solid in nature. Solid suspensions may be formed from crystalline or amorphous materials, or a combination thereof, under appropriate conditions. However, it may be the case that solid suspensions of the invention are formed from crystalline materials. Solid suspensions are advantageous relative to powders, gel networks and glasses, as they can be injected, aiding surgical delivery, particularly when a defect area may be difficult to access. With regards to a gel network or gel based material, an activation step would be required, which would add preparation time and an added level of complexity for the delivery of the device. In addition, the use of gels inherently involves a dynamic gelation stage which can be detrimentally affected by temperature and concentration of components. As such, gels would be difficult to control in a surgical situation because performing the gelation step prior to surgical delivery can reduce the injectability of gel materials. Specifically, the rheological properties of gels can be detrimentally affected by applying shear forces which can irreversibly damage a gel network.

The group II cations may be provided as sulfates, phosphates, carbonates, oxides, silicates, peroxides, sulfides and/or halides. Often they will be provided as phosphates, often hydroxyapatites. It is generally the case that where more than one group II cation is present, the sources of these are independent, such that each cation will be provided from a different source (although the counter-ion for each source will sometimes be the same such that calcium phosphate and strontium phosphate may be independently provided). Although dicationic compounds may be used (such as ion-exchanged dicationic calcium-strontium hydroxyapatite), generally if a combination of calcium and strontium is required, this will be provided as a calcium compound and a separate strontium compound.

Where present, the calcium cations in the solid suspension may be provided as a calcium compound selected from calcium sulfate anhydrite (CaSO₄), calcium sulfate hemihydrate (CaSO₄.0.5H₂O), calcium sulfate dihydrate (CaSO₄.2H₂O), monocalcium phosphate (Ca(H₂PO₄)₂), anhydrous dicalcium phosphate (CaHPO₄), tricalcium phosphate (Ca₃(PO₄)₂), tetracalcium phosphate (Ca₄(PO₄)₂O), octacalcium phosphate (Ca₈H₂(PO₄)₆.5H₂O), hydroxyapatite (Ca₅(PO₄)₃(OH)), calcium carbonate (CaCO₃), calcium oxide (CaO) and calcium silicate (CaSiO₃) or combinations thereof. These compounds have been found to offer good biocompatibility and release of calcium cations during bone regeneration. Often the calcium compound will comprise a phosphate, which may be selected from monocalcium phosphate, anhydrous dicalcium phosphate, tricalcium phosphate, tetracalcium phosphate, octacalcium phosphate, hydroxyapatite or combinations thereof, most often the calcium compound will comprise hydroxyapatite, often the calcium compound will consist of or consist essentially of hydroxyapatite. This is for biocompatibility reasons. As such, the solid suspension may comprise a calcium compound which may be hydroxyapatite.

In the solid suspension the group II cation compound, such as the calcium compound, may be present in crystalline form, often the group II cation compound (whether or not crystalline) may be present as a nano-compound. As used herein the term “nano” is intended to denote particles of a mean particle diameter (widest point) in the range 1 nm-100 nm, often 5 nm-75 nm, 25 nm-50 nm or 50 nm-100 nm. Within these ranges the nano-compound, often nano-hydroxyapatite, closely resembles the scale found in natural human bone.

Often, where present, the strontium cations are provided as a strontium compound selected from strontium sulfate (SrSO₄), strontium phosphate (Sr₃(PO₄)₂), strontium carbonate (SrCO₃), strontium oxide (SrO), strontium peroxide (SrO₂), strontium phosphide (Sr₃P₂), strontium sulfide (SrS), strontium chloride (SrCl₂), strontium-substituted hydroxyapatite (Sr₅(PO₄)₃(OH)) and strontium ranelate (C₁₂H₆N₂O₈SSr₂) or combinations thereof. Often, the strontium compound comprises strontium-substituted hydroxyapatite. As such, the solid suspension may comprise a strontium compound which may be strontium-substituted hydroxyapatite.

The ratio of calcium:other group II cations (such as strontium cations) in the suspension may be in the range 0:100-99:1, or 1:99-70:30. As such, where the ratio is 0:100 calcium:other group II, the calcium cations may be absent. The presence of strontium has been found to enhance the antimicrobial and osteogenic properties of the solid suspension. Accordingly, a source of group II cations will often be strontium, often strontium-substituted hydroxyapatite, often the group II cations will be entirely supplied by the provision of strontium-substituted hydroxyapatite (100% strontium-substituted hydroxyapatite or a ratio of calcium:strontium of 0:100). Alternatively, there may be as little as 1% strontium, which may be provided as strontium-substituted hydroxyapatite, the remaining group II cation optionally being provided in the form of a group II cation substituted-hydroxyapatite. Often this will comprise calcium hydroxyapatite. Often the ratio of calcium:strontium is greater than or equal to 70:30 (30% or more strontium). Even where the group II cation is only 30% strontium, the suspensions have been found to offer excellent antimicrobial and osteogenic properties.

The solid suspension will comprise zinc oxide, often the zinc oxide will be of mean particle size in the range 0.01 μm-100 μm, often in the range 0.1 μm-50 μm or 0.2 μm to 10 μm. At sizes in this range the zinc oxide has been found to mix well with the solid suspension forming good homogeneous pastes. Zinc oxide is generally present at least partially, often totally, in crystalline form in the solid suspension. The presence of zinc oxide in crystalline form is advantageous, as crystalline zinc oxide has added antibacterial activity over ionic zinc. In glasses, the components are combined to form a non-crystalline amorphous solid, meaning that any antibacterial action arising from a zinc component would be reliant on ionic zinc incorporated into the glass network. Therefore, it is suggested that a zinc oxide containing glass would exhibit an inferior antibacterial activity to the solid suspensions of the invention.

Often the solid suspension will comprise in the range 20-60 wt % solid, often 30-45 wt % solid, or 35-45 wt % solid; the remaining 40-80 wt % being a liquid component. Often the solid suspension will comprise in the range 55-70 wt % or 55-65 wt % liquid component. At these levels of solid, the solid suspension will be in the form of a paste, namely a semi-fluid composition. This ensures that the solid suspension is easy to manipulate, in particular being injectable to an implantation location, without being unduly fluid such that it will remain at the implantation location after application, without flowing away.

It may be the case that the liquid component comprises water, for its ready accessibility and biocompatibility, often deionised water.

The use of zinc oxide can be beneficial as zinc oxide can generate reactive oxygen species, which contribute to the antimicrobial effect of the suspension. Typically, the zinc oxide will be present in the range 0.25 wt %-5.0 wt % of the solid suspension, often in the range 0.5 wt % to 3.0 wt %, often 1.0 wt %-2.0 wt %. At these ranges good antimicrobial activity is observed.

The solid suspension may be for use in the treatment of bone defects arising from osteoporosis, osteoarthritis or other bone degenerative diseases. In such cases, the solid suspension may be (or is to be) administered to the bone defect site by injection.

The solid suspension may be at least partially bioresorbable, allowing for dissolution of any bone graft formed from the solid suspension as bone regrowth occurs. Often it will be the case that the solid suspension is for use primarily in bone regeneration, rather than in the repair of bone defects although this may be an additional use, often the solid suspension will be used in dental applications, or in orthopaedic or spinal bone regeneration.

In a second aspect of the invention there is provided a bone graft which may be bioresorbable, comprising the solid suspension of the first aspect of the invention.

In a third aspect of the invention there is provided a method of preparing a solid suspension for use in bone regeneration and/or the repair of bone defects comprising: mixing at least one group II metal cation with zinc oxide, wherein where there is only one group II metal cation this is strontium.

Often the solid suspension will be the solid suspension of the first aspect of the invention. It may be that the group II cations are calcium and/or strontium cations, in such cases the source of calcium cations may be a calcium compound such as hydroxyapatite and the source of strontium ions may be a strontium compound such as strontium-substituted hydroxyapatite.

In a fourth aspect of the invention there is therefore provided a method of forming a bone graft comprising delivering a solid suspension of the first aspect of the invention to an implantation location. The bone graft may be bioresorbable. Often the implantation location is in the human or animal body, often the implantation location is orthopaedic, spinal or dental.

In a fifth aspect of the invention there is provided the use of the solid suspension of the first aspect of the invention in bone regeneration and/or the repair of bone defects. Often the bone regeneration will be cosmetic, although use in the treatment of conditions such as osteoporosis and osteoarthritis is also envisaged. This is particularly the case where the bone regeneration is dental.

There is therefore provided an at least partially resorbable solid suspension for use in bone regeneration comprising in the range 35-45 wt % solid, the solid comprising:
calcium cations provided as a calcium compound comprising nano-hydroxyapatite,
strontium cations provided as a strontium compound comprising strontium-substituted hydroxyapatite, and
zinc cations, provided by crystalline zinc oxide of particle size in the range 0.2 μm to 10 μm wherein the zinc oxide may be present in the range 0.25 to 5.0 wt % of the solid suspension. A ratio of calcium:strontium cations may be in the range 0:100-70:30.

Unless otherwise stated, each of the integers described may be used in combination with any other integer as would be understood by the person skilled in the art. Further, although all aspects of the invention preferably “comprise” the features described in relation to that aspect, it is specifically envisaged that they may “consist” or “consist essentially” of those features outlined in the claims. In addition, all terms, unless specifically defined herein, are intended to be given their commonly understood meaning in the art.

Further, in the discussion of the invention, unless stated to the contrary, the disclosure of alternative values for the upper or lower limit of the permitted range of a parameter, is to be construed as an implied statement that each intermediate value of said parameter, lying between the smaller and greater of the alternatives, is itself also disclosed as a possible value for the parameter.

In addition, unless otherwise stated, all numerical values appearing in this application are to be understood as being modified by the term “about”.

In order that the invention may be more readily understood, it will be described further with reference to the figures and to the specific examples hereinafter.

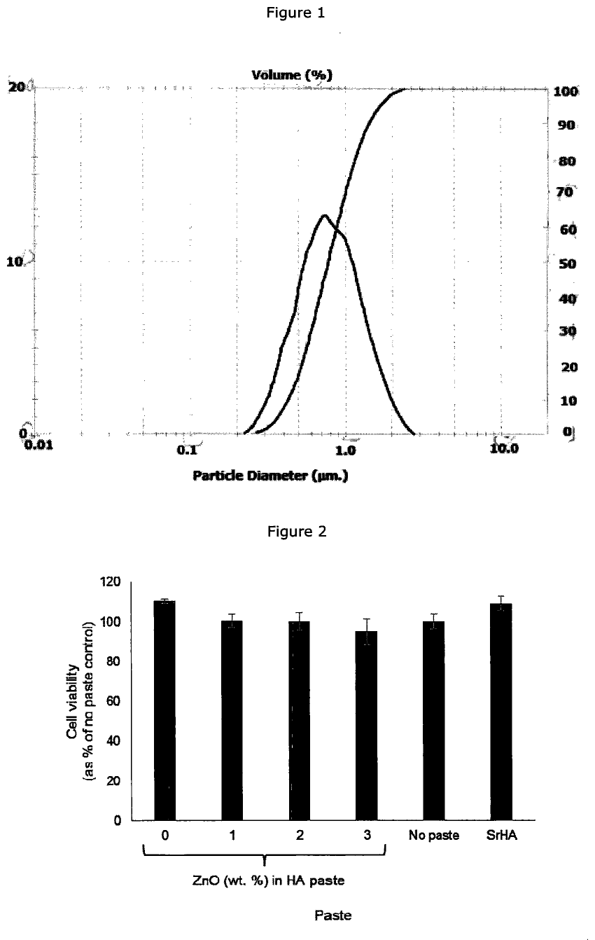

FIG. 1 shows the particle size distribution of zinc oxide used in the test examples;

FIG. 2 is a graph illustrating the biocompatibility of the pastes/suspensions tested. Calcium hydroxyapatite pastes containing 0, 1, 2 or 3 wt % zinc oxide, no paste and strontium-substituted hydroxyapatite paste were tested;

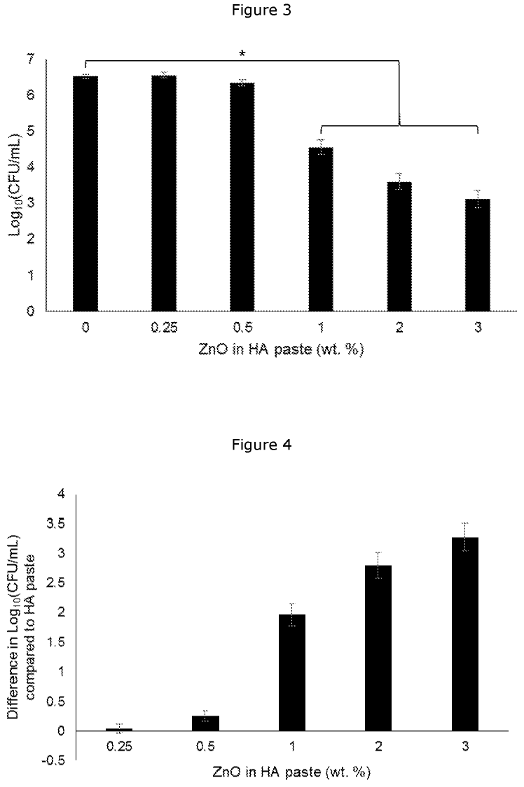

FIG. 3 is a graph illustrating the antibacterial effect of increasing concentration of zinc oxide in a hydroxyapatite paste. Shown are the number of viable S. aureus after 24 h. Error bars±S.E.M. Significance: *p<0.05;

FIG. 4 is a graph illustrating the reduction in the number of viable S. aureus after 24 h at different concentrations of zinc oxide in hydroxyapatite, based on the data of FIG. 3. Error bars±S.E.M; and

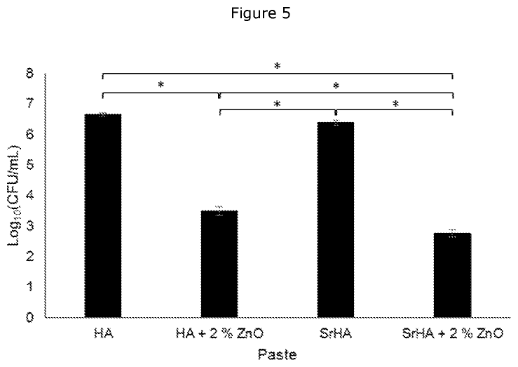

FIG. 5 is a graph illustrating the antibacterial effect of each paste of hydroxyapatite, hydroxyapatite+2 wt % zinc oxide, strontium-substituted hydroxyapatite, and strontium-substituted hydroxyapatite+2 wt % zinc oxide. Shown are the number of viable S. aureus attached to HA and SrHA pastes containing 2 wt % ZnO after 24 h. Error bars±S.E.M. Significance: *p<0.05.

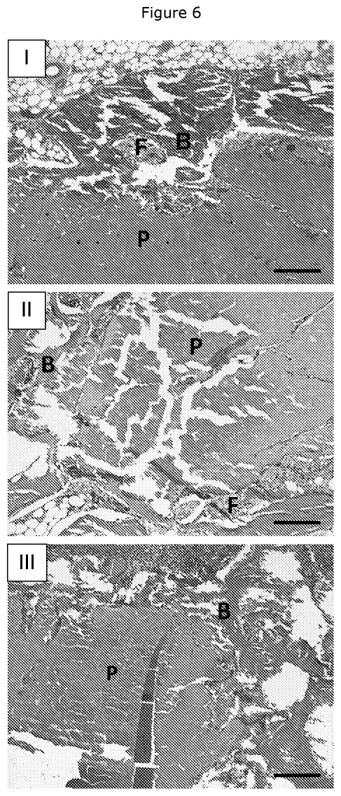

FIG. 6 is haematoxylin and eosin stained histology sections of defect sites from rabbit femoral condyle implantation of 100 wt. % strontium-substituted hydroxyapatite+2 wt. % zinc oxide paste, which show the paste in contact with bone (i) 6 weeks, (ii) 12 weeks, and (iii) 19 weeks after implantation. In FIG. 6, P represents paste, B represents bone, F represents fibrous tissue, and the scale bar represents 200 μm.

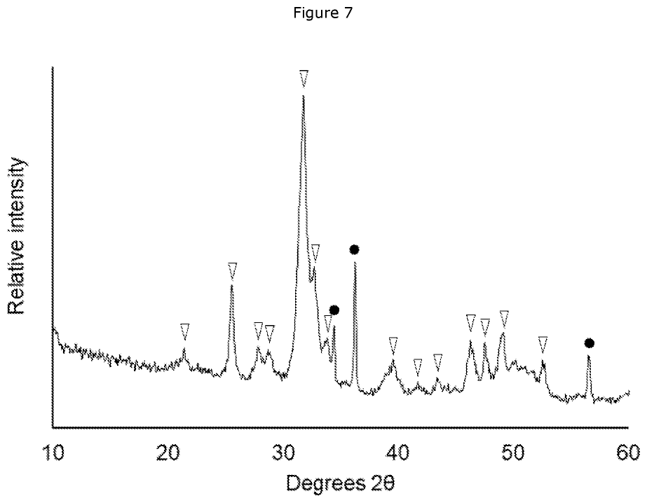

FIG. 7 is an X-ray diffraction pattern of a composition containing 30 wt. % strontium-substituted hydroxyapatite+4 wt. % zinc oxide, wherein “∇” represents 35 wt % strontium-substituted hydroxyapatite (ICDD PDF 04-016-3586), and “●” represents zinc oxide (ICDD PDF 5-664).

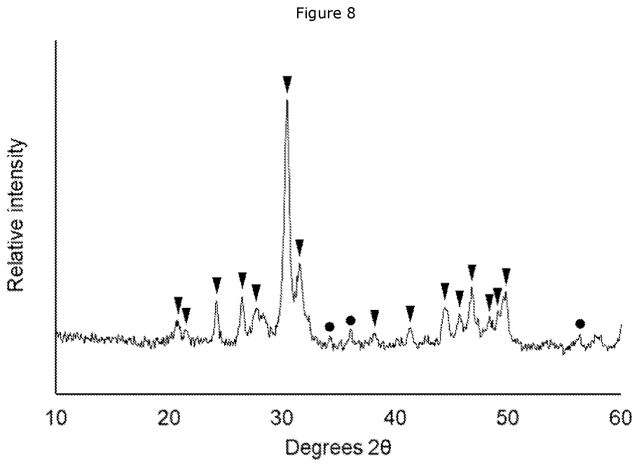

FIG. 8 is an X-ray diffraction pattern of a composition containing 100 wt. % strontium-substituted hydroxyapatite+2 wt. % zinc oxide, wherein “▾” represents 100 wt % strontium-substituted hydroxyapatite (ICDD PDF 33-1348), and “●” represents zinc oxide (ICDD PDF 5-664).

### EXAMPLES

Paste Manufacture

Pastes were prepared by mixing crystalline zinc oxide powder with hydroxyapatite (HA) slurries. Depending on the example, the hydroxyapatite slurries may be calcium, strontium or combinations thereof in the cationic ratios discussed. A portion of the water content was then evaporated in a drying oven resulting in a paste consistency. The amount of zinc oxide was calculated as a percentage of the final HA paste (HA solids+water) with the residual solid content of the HA paste being in the region of 38-40 wt %.

Test Methods

Antibacterial Evaluation

The ability of the pastes to prevent bacterial colonisation on the paste surface was assessed using a biofilm initialisation model. The pastes were tested for antibacterial activity against a clinical isolate of Staphylococcus aureus (S235). The attachment and survival of bacteria to the surface of the pastes was assessed after a 24 h incubation.

10 mm (±1 mm) lengths of sterile pastes extruded from a standard luer lock syringe were placed into sterile 1 ml Eppendorfs, with 3 samples prepared for each paste. 500 μL of sterile PBS (phosphate buffered saline) was added to each Eppendorf. A bacterial suspension of OD 0.05 was prepared in PBS and 500 μL of bacterial suspension was added to the Eppendorf tubes containing paste (n=3). The tubes were incubated at 37° C. for 20 h. The PBS was removed from all tubes and the paste lengths were washed twice with 1 ml PBS. To assess the number of viable bacteria attached to the paste after the overnight incubation the pastes were suspended and serial dilutions were plated on agar plates to allow for colonies to be counted. In detail, the washed pastes were suspended in 1 ml PBS using a vortex. Two serial dilutions were then performed per sample in PBS and the following dilutions were plated up by placing 10 μL sample onto BHI (brain heart infusion) agar plates: 1 in 10⁰, 10¹, 10², 10³ and 10⁴. The plates were incubated overnight at 37° C. and the colonies were counted at an appropriate dilution where single colonies could easily be identified.

Statistical analysis was carried out on the log numbers from the experiment using SPSS software. The statistical analysis firstly involved a one way ANOVA test. The post hoc test was then selected based on the homogeneity of the variance between the experimental groups. Levene's test was used to determine the homogeneity of the variances. If the variances were not significantly different from each other, Tukey's multiple comparisons test was used. If the variances were significantly different from each other, Games-Howell comparison was applied.

Biocompatibility of Pastes

The biocompatibility of the pastes was investigated by seeding 50,000 MG63 cells per well with 1 mL basal media (made up of the following v/v %: α-MEM supplemented with 10% foetal calf serum, 1% l-alanyl-l-glutamine, 1% penicillin-streptomycin, 1% non-essential amino acids) in a 24 well plate. The cells were incubated at 37° C., 5% CO₂ for 24 h after which the media was removed and 0.9 mL media was added. Permeable Millicell® hanging inserts (0.4 μm pore size, Merck Millipore) were then placed in each well and 0.2 mL complete media was added inside each insert. 0.1 mL paste was then added to each insert in triplicate and the plates were incubated at 37° C., 5% CO₂ for an additional 24 h. After the incubation the inserts were removed and the cells were imaged using light microscopy. The media was removed from the cells and 0.5 mL of a 10% (v/v) PrestoBlue® solution in complete media was added to each well. PrestoBlue® solution was also added in triplicate to empty wells as a control to subtract from the fluorescence values obtained. The samples were incubated at 37° C., 5% CO₂ until appropriate colour change was observed. At each time point 0.2 mL solution was placed into a 96 well plate. The fluorescence of the solutions was measured using a plate reader with an excitation wavelength of 535 nm and an emission wavelength of 590 nm. The same statistical approach was used as described above for the antibacterial testing.

Test Results

Particle Size Distribution of Zinc Oxide

The particle size distribution of the crystalline zinc oxide used in these tests was as shown in Tables 1 and 2 below. FIG. 1 illustrates this in graphical form.

TABLE 1
Particle size distribution

| Under | 0.4 μm | 0.6 μm | 0.8 μm | 1 μm | 3 μm |
|---|---|---|---|---|---|
| % | 5-15 | 20-40 | 45-60 | 60-80 | 100 |

TABLE 2
Particle size distribution

| | D10 | D50 | D90 |
|---|---|---|---|
| μm | 0.30-0.45 | 0.70-0.85 | 1.20-1.70 |

Biocompatibility of Pastes

FIG. 2 shows the excellent biocompatibility of the suspensions. Specifically it shows good cell viability (MG63 cells) with calcium hydroxyapatite pastes containing 0, 1, 2 and 3 wt % zinc oxide, with strontium-substituted hydroxyapatite paste and in the absence of paste. It is clear that cell viability is not significantly affected by the presence of the suspensions.

Antibacterial Activity of Zinc Oxide

FIGS. 3 and 4 show the antibacterial activity of hydroxyapatite pastes containing zinc oxide in a range of concentrations. It can be seen that the presence of zinc oxide enhances the antibacterial properties of the hydroxyapatite, this enhancement becoming significant when the zinc oxide is present at a level above 0.25 wt % of the solid.

Antibacterial Activity of Zinc Oxide Combined with Strontium

The antibacterial activity of the following four compositions was measured and compared:
1. hydroxyapatite,
2. hydroxyapatite+2 wt % of the solid suspension zinc oxide,
3. strontium-substituted hydroxyapatite, and
4. strontium-substituted hydroxyapatite+2 wt % of the solid suspension zinc oxide

As can be seen from FIG. 5, and discussed above, the addition of zinc oxide alone enhances the antibacterial properties of hydroxyapatite. FIG. 5 also shows that the presence of strontium enhances the antibacterial properties of hydroxyapatite. Unexpectedly, however, where both zinc oxide and strontium are present a synergistic effect is observed. Specifically, it has been shown that the combination of the zinc and strontium has a greater antibacterial effect in terms of the reduction of bacterial numbers than would be expected from an additive effect of each of the zinc and strontium compounds alone (i.e. the number of viable bacteria attached to the SrHA+2% ZnO is significantly lower than the number of viable bacteria attached to SrHA and HA+2 wt % ZnO pastes). Therefore, the zinc oxide and strontium appear to be working together, in synergy, to provide an antibacterial effect.

It would be appreciated that the methods of the invention are capable of being implemented in a variety of ways, only a few of which have been illustrated and described above.

In Vivo Bone Regeneration

FIG. 6 shows haematoxylin and eosin stained histology sections of defect sites from rabbit femoral condyle implantation of 100 wt. % strontium-substituted hydroxyapatite+2 wt. % zinc oxide paste, which show the paste in contact with bone (i) 6 weeks, (ii) 12 weeks, and (iii) 19 weeks after implantation. These results show in vivo bone regeneration.

Crystallinity

FIG. 7 shows the X-ray diffraction pattern of a composition containing 30 wt. % strontium-substituted hydroxyapatite+4 wt. % zinc oxide. The peaks are indicative of the presence of a mixture of strontium-substituted hydroxyapatite and crystalline zinc oxide.

FIG. 8 shows the X-ray diffraction pattern of a composition containing 100 wt. % strontium-substituted hydroxyapatite+2 wt. % zinc oxide. As with FIG. 7, the peaks are indicative of the presence of a mixture of strontium-substituted hydroxyapatite and crystalline zinc oxide.

1. A solid suspension for use in bone regeneration and/or the repair of bone defects, comprising a source of at least one group II metal cation and a source of zinc cations, wherein the source of zinc cations comprises zinc oxide and wherein where there is only one group II metal cation this is strontium.

2. A solid suspension according to claim 1, wherein the group II metal cations are selected from calcium, strontium, magnesium and combinations thereof.

3. A solid suspension according to claim 2, wherein the group II metal cations are selected from calcium, strontium and combinations thereof.

4. A solid suspension according to claim 2 or claim 3, wherein the calcium cations are provided as a calcium compound selected from calcium sulfate anhydrite (CaSO₄), calcium sulfate hemihydrate (CaSO₄.0.5H₂O), calcium sulfate dihydrate (CaSO₄.2H₂O), monocalcium phosphate (Ca(H₂PO₄)₂), dicalcium phosphate anhydrous (CaHPO₄), tricalcium phosphate (Ca₃(PO₄)₂), tetracalcium phosphate (Ca₄(PO₄)₂O), octacalcium phosphate (Ca₈H₂(PO₄)₆.5H₂O), hydroxyapatite (Ca₅(PO₄)₃(OH)), calcium carbonate (CaCO₃), calcium oxide (CaO) and calcium silicate (CaSiO₃) or combinations thereof.

5. A solid suspension according to claim 4, wherein the calcium compound comprises a phosphate.

6. A solid suspension according to claim 5, wherein the calcium compound comprises hydroxyapatite.

7. A solid suspension according to any preceding claim, wherein the strontium cations are provided as a strontium compound selected from strontium sulfate (SrSO₄), strontium phosphate (Sr₃(PO₄)₂), strontium carbonate (SrCO₃), strontium oxide (SrO), strontium peroxide (SrO₂), strontium phosphide (Sr₃P₂), strontium sulfide (SrS), strontium chloride (SrCl₂), strontium-substituted hydroxyapatite (Sr₅(PO₄)₃(OH)) and strontium ranelate (C₁₂H₆N₂O₈SSr₂) or combinations thereof.

8. A solid suspension according to claim 7, wherein the strontium compound comprises strontium-substituted hydroxyapatite.

9. A solid suspension according to any of claims 4 to 8, wherein the hydroxyapatite comprises nano-hydroxyapatite.

10. A solid suspension according to any of claims 4 to 9, comprising a ratio of calcium:strontium cations in the range 0:100-99:1.

11. A solid suspension according to any preceding claim, wherein the zinc oxide is of mean particle size in the range 0.01 μm-100 μm.

12. A solid suspension according to any preceding claim, further comprising a liquid component comprising water.

13. A solid suspension according to any preceding claim, comprising in the range 20-60 wt % solid.

14. A solid suspension according to any preceding claim, comprising zinc oxide in the range 0.25-5.0 wt % of the solid suspension.

15. A solid suspension according to any preceding claim, wherein the solid suspension is at least partially bioresorbable.

16. A solid suspension according to any preceding claim, for use in bone regeneration and/or in the repair of bone defects.

17. A solid suspension according to any preceding claim, for use in the treatment of bone defects arising from osteoporosis, wherein the solid suspension is to be administered to the bone defect site by injection.

18. A solid suspension according to any of claims 1 to 17, for use in the treatment of bone defects arising from osteoarthritis, wherein the solid suspension is to be administered to the bone defect site by injection.

19. A bone graft, comprising the solid suspension of any preceding claim.

20. A method of preparing a solid suspension according to any of claims 1 to 18, comprising: mixing at least one group II metal cation with zinc oxide, wherein where there is only one group II metal cation this is strontium.

21. A method of forming a bone graft comprising delivering a solid suspension of any of claims 1 to 18 to an implantation location.

22. A method according to claim 21, wherein the implantation location is in the human or animal body.

23. A method according to claim 21 or claim 22, wherein the implantation location is orthopaedic, spinal or dental.

24. A use of the solid suspension of any of claims 1 to 18 in bone regeneration and/or in the repair of bone defects.

25. A use according to claim 24, in orthopaedic or spinal bone regeneration.

26. A use according to claim 24, in the treatment of bone defects arising from osteoporosis or osteoarthritis.

27. A use according to claim 24, wherein the bone regeneration is cosmetic.

28. A use according to claim 27, wherein the cosmetic bone regeneration is dental.
